(19) United States
(12) Patent Application Publication
(10) Pub. No.: US 2019/0387987 A1
(43) Pub. Date: Dec. 26, 2019

(54) Method For Analysing Cardiovascular Parameters Of An Individual

(71) Applicant: WITHINGS, Issy Les Moulineaux (FR)

(72) Inventors: Antoine MERLOT, Paris (FR); David CAMPO, Boulogne Billancourt (FR); Virginie VISSAC, Boulogne Biilancourt (FR); Haïkel BALTI, Vitry Sur Seine (FR)

(73) Assignee: WITHINGS, Issy Les Moulineaux (FR)

(21) Appl. No.: 16/480,433

(22) PCT Filed: Mar. 13, 2018
(86) PCT No.: PCT/FI2018/050177

§ 371 Date: Jul. 24, 2019

(30) Foreign Application Priority Data
Mar. 15, 2017 (EP) 17161116.3

(51) Int. Cl. A61B 5/021; A61B 5/022; A61B 5/0402

(52) U.S. Cl. CPC A61B 5/02125; A61B 5/6823; A61B 5/0402; A61B 5/02233

(57) ABSTRACT

A method to collect cardiovascular data relating to an individual user (U), in a system comprising an acoustic sensor (4) configured to be coupled to the chest of the user, an arm band (2) having an inflatable bladder (53) configured to be placed around at a predefined position around a upper limb of the user, for example the left arm (BG), a pneumatic unit with at least a pump (7) and a pressure sensor (61), configured to inflate and deflate the inflatable bladder, an arterial blood path (P) being defined from the heart to the left arm (BG) of the user,the method comprising a set of steps named PTT procedure: /A1/-acquiring acoustic signals at the acoustic sensor, /A2/-acquiring pressure signals at the pressure sensor, representative of pressure occurring at the brachial artery, /S0/-inflate the bladder at a predetermined pressure (PT1) /S1/-determining aortic valve closing instant T1(k) from acoustic signals, /S2/-determining subsequently, from pressure signals, a characteristic point (M2) of the pressure signal curve occurring at instant T2(k), /S3/-calculate a time difference, defined as ΔT(k)=T2(k)−T1(k) Repeating, for each heartbeat, steps S1 to S3 until a stop criterion(SC) is met.

### FIELD OF THE DISCLOSURE

The present disclosure relates to, methods for analysing cardiovascular parameters of an individual and devices used to carry out such methods.

More particularly, it relates to a method designed to assess parameters of the type “pulse transit time”, these parameters being determined from acoustic signals and/or electrical signals emitted by the heart of such individual, and from responsive signals sensed at a blood circulation sensor, which may be a blood pressure sensor or other type of sensor. Likewise for one instance this kind of method, there may be used an inflatable bladder comprised in a blood pressure cuff.

### BACKGROUND OF THE DISCLOSURE

There is known devices that combine arterial pressure sensing means and electrocardiogram sensing means, of the type for example disclosed in document US20160235325, in which some attempts have been done to assess pulse transit time from ECG signal. However, such method shows inaccuracy shortcomings since ECG signal fails to accurately reflect the mechanical activity of the heart. Furthermore, acquiring pressure waves at the wrist is not fully adequate to accurately detect some trouble located in the arterial network close to the heart.

Therefore, the inventors have identified a need to enhance functionalities provided by such methods and improve their accuracy.

SUMMARY OF THE DISCLOSURE

According to one aspect of the present disclosure, there is disclosed a method to collect cardiovascular data relating to an individual user (U), in a system comprising an acoustic sensor (4) configured to be coupled to the chest of the user, a blood circulation sensor configured to be placed at a predefined position at the user's body, an arterial blood path (P) being defined from the heart to said predefined position, the method comprising a set of steps named PTT procedure
/A1/—acquiring acoustic signals at the acoustic sensor,
/A2/—acquiring of blood circulation signals at the blood circulation sensor, said blood circulation signals being representative of instantaneous blood circulation parameters prevailing at the predefined position,
/S1/—determining aortic valve closing instant T1(k) from acoustic signals,
/S2/—determining subsequently, from blood circulation signals, a characteristic point (M2) occurring at instant T2(k),
/S3/—calculate a time difference, defined as ΔT(k)=T2(k)−T1(k)
/Sloop/ repeating, for each heartbeat, steps /S1/ to /S3/ until a stop criterion (SC) is met.

Thereby, the electronic controller acquires acoustic signals, sensed by the acoustic sensor, in timely conjunction with blood circulation parameters at the predefined position, to determine therefrom for example a Pulse Transit Time (PTT) from the aortic valve to the artery location of interest.

The term “predefined position” should be understood as a predefined location at an artery vessel where the heart pulses reflect. The artery location of interest is not necessarily at the vicinity of the skin, it can be located in depth into a user's limb.

It should be understood that the so-called predefined position can be located at the left arm, but it could also be somewhere else on the left arm, e.g. on the forearm, on the wrist, somewhere else on the right arm of the user, including the wrist; it is not excluded to implement the proposed method with pressure signals taken on a lower limb of the user. Of course, calculation parameters to be used are to be adapted in particular to the length of the arterial blood path (P) for each case.

The inventors have found that acoustic signals provide a good time marker for start time in order to perform calculation of Pulse Transit Time (PTT). Particularly, the closure of the aortic valve produces a sufficient noise to be properly captured.

The term “blood circulation parameters” can designate the blood local pressure, the blood local speed, or even an image or an aggregate parameter representative of both pressure and speed, prevailing at the predefined position.

The term “blood circulation sensor” can designate a pressure sensor or a Doppler effect sensor or another type of sensor.

In one implementation, the system may comprise means for exerting pressure around a limb of the user, at the predefined position, and the blood circulation sensor may be a pressure sensor, wherein blood fluid circulation parameters signals are pressure signals, and wherein the method comprises, prior to step /S1/:
/S0/—exert a predetermined pressure (PT1)
And at step /S2/ the characteristic point (M2) is determined from a pressure signal curve. With pressure signals, the method works well even though the artery vessel is deep below the skin.

In one implementation, the means for exerting pressure is a band (2) having an inflatable bladder (53) configured to be placed at the predefined position, a pneumatic unit with at least a pump (7), the pressure sensor being configured to be fluidly connected to the inflatable bladder,

While aortic valve closure can be taken as a first time marker and, further, the corresponding reflection/response in the pressure signal at bladder can be taken as a relevant second time marker.

In one implementation, the predefined position is at the left arm (BG) of the user, the band (2) is an arm band and is configured to be placed around the left arm of the user. It should be noted that, in the case of the left arm, the blood path at interest for assessing the Pulse Transit Time (PTT) is rather short namely less than 40 cm and concentrates on the main arterial network starting from the heart, which enables to detect potential problems affecting aorta or other portion of the main arterial network leading from the heart to the arm, (under this perspective the atm can be a better location than the wrist or a leg).

Thereby, the electronic controller acquires pressure wave signals to determine therefrom for example a Pulse Transit Time (PTT) from the aortic valve to the brachial artery.

According to one particular option, there is disclosed a method to collect cardiovascular data relating to an individual user (15), in a system comprising an acoustic sensor (4) configured to be coupled to the chest of the user, an arm band (2) having an inflatable bladder (53) configured to be placed at a predefined position around a limb of the user, for example at the left arm (BG), a pneumatic unit with at least a pump (7) and a pressure sensor (61), configured to inflate and deflate the inflatable bladder, an arterial blood path (P) being defined from the heart to the predefined position, for example the left arm (BG) of the user, the method comprising a set of steps named PTT procedure:
A1—acquiring acoustic signals at the acoustic sensor,
A2—acquiring pressure signals at the pressure sensor, representative of pressure prevailing at the brachial artery,
/S0/—inflate the bladder at a predetermined pressure (PT1)
/S1/—determining aortic valve closing instant T1(k) from acoustic signals,
/S2/—determining subsequently, from pressure signals, a characteristic point (M2) of the pressure signal curve occurring at instant T2(k),
/S3/—calculate a time difference, defined as ΔT(k)=T2(k)−T1(k)
/Sloop/ repeating, for each heartbeat, steps S1 to S3 until a stop criterion (SC) is met.

The arterial blood path of interest is therefore mainly focused on the central arterial distribution network; this improves the detection of potential problems affecting aorta or other portion of the main arterial network leading from the heart to the tell arm.

The blood path at interest for assessing the Pulse Transit Time (PTT) is rather short namely less than 40 cm and concentrates on the main arterial network starting from the heart, which enables to detect potential problems affecting aorta or other portion of the main arterial network leading from the heart to the left arm.

According to one particular option, the characteristic point (M2) of the pressure signal curve is defined as a succession of a maximum apex (M1) and a minimum apex (M2), said instant T2(k) being defined as the instant when said minimum apex occurs.

Faithfull and relevant time marker corresponding to arrival at the arm of the effect of the closure of aortic valve is thus determined.

According to one particular option, the system comprises a set of contact electrodes (3) for electrocardiographic sensing, configured to be brought in contact with the skin of the user U, the method comprising the steps:
/A3/—acquiring ECG signals at the contact electrodes,
/S10/—determining a characteristic QRS signal from ECG signals as a synchronization signal (T0) reflecting heartbeat.

This helps to synchronize the analysis of acoustic and blood circulation Signals.

According to one particular option, the aortic valve closing instant T1(k) is defined as a second significant sound (B2) of the heartbeat.

Faithfull/relevant time marker corresponding to the closure of aortic valve is thus determined.

According to one particular option, wherein at step /S1/, the aortic valve closing instant T1(k) is determined as follows:
/S11/—identifying a first significant sound (B1) of the heartbeat, reflecting mitral valve closing, just following QRS signal at, instant T0,
/S12/—identifying a second significant sound (B2) of the heartbeat, reflecting aortic valve closing, and record said second significant sound (B2) as instant T1(k).

This provides a simple and reliable method to determine instant T1(k).

According to one particular option, a significant sound is defined whenever a instantaneous power of the acoustic signals exceeds a predetermined threshold (BS). Noises can thus be disregarded.

According to one particular option, the method comprises, before the PTT procedure, a preliminary set of steps named Blood Pressure procedure comprising the steps:
/Ph1/—start inflating the bladder,
/Ph2/—stop inflating the bladder (when no more pressure wave is identified),
/Ph3/—start deflating the bladder,

meanwhile are performed the following steps:
/PhS/—determining Systolic Blood pressure (PTS), during inflating phase and/or deflating phase
/PhD/—determining Diastolic Blood pressure (PTD), during inflating phase and/or deflating phase

This provides a blood pressure reference prior to carry out PTT procedure; abortion of PTT can be decided if BP procedure is incorrect.

According to one particular option, prior to steps /S1/ to /S3/, the predetermined pressure PT1, providing pressurization for PTT procedure, is calculated with reference to the diastolic pressure (PTD) determined at step PhD.

PT1 is therefore user dependent.

According to one particular option, the predetermined pressure PT1 can be defined as a function of PTD, for example with a value below PTD this difference defined by a predefined offset; in other words, PT1 can be equal to PTD−PTof; with PTof for example equal to 10 mmHg (10 Torr).

According to one particular option, the stop criterion SC is met after N heartbeats for which steps S1 to S3 were carried out properly, N being comprised between 4 and 10.

PTT procedure can be short. Time duration can be defined by user or according to a quality index criteria. Respiration/Breathing or other disturbances can be compensated for.

According to one particular option, at steps /A1/ and/or /A2/, acoustic signals and/or pressure signals are digitalized are further filtered with a band pass filter having a passing band range of [0.5 Hz-1 kHz].

Continuous components and noises can thus be eliminated,

According to one particular option, during PTT procedure, pump is nor energized and a bleeder valve comprised in the pneumatic unit is not energized.

No noise from the device itself: no disturbance on acoustic signals.

According to one particular option, the method may further comprise:
/S41/—calculate an average value ΔTav of ΔT(k), for k=j to j+N (with N being the number of heartbeats with effective PTT measurement),
/S42/—calculate a Pulse Wave Velocity (PWV) defined as PWV(k)=length(P)/ΔTav,
/S5/—assess therefrom an arterial stiffness (AS) of the user.

This provides a reliable rating as over several cycles.

According to one particular option, the height (UH) of the user is taken into account at step /S42/, namely length(P)=F1 (UH)

This provides a personal index, which is relevant whatever the length of the blood path (which depends on the height of the user at first order).

According to one particular option, age (UA), gender (UG), and weight (UW), of the user is additionally taken into account at step /S42/, namely length(P)=F2 (UH,UA,UG,UW).

A cardiovascular index can be refined to faithfully reflect the arterial stiffness, irrespective of user profile particulars.

According to one particular option, subsequent evaluations of Pulse Wave Velocity are recorded for a particular user to form a history curve, and a deviation in said curve is notified to said particular user.

The change in user habits can be detected by this means; said change can relate to food intake, physical exercise, initial phase of a disease etc.

The present disclosure is also directed to a system, device or apparatus configured to carry out the above-mentioned methods and functionalities.

The present disclosure is also directed to computer readable medium encoded with instructions that, when executed by a computer, cause performance of a method as described above.

### BRIEF DESCRIPTION OF THE DRAWINGS

Other features and advantages of the disclosure appear from the following detailed description of one of its embodiments, given by way of non-limiting example, and with reference to the accompanying drawings, in which:

FIG. 1 illustrates a general overview of a device according to the present disclosure in a use configuration,

FIG. 2 shows a diagrammatic sectional view of the device in place on the left arm of the user, and adjacent to the chest, for the generically defined embodiment,

FIG. 3 illustrates a mechanical configuration of the device in an open configuration, according to a first embodiment,

FIG. 4 shows a diagrammatic sectional view of the device in place on the left arm of the user, according to the embodiment shown in FIG. 3,

FIG. 5 shows another diagrammatic sectional view of the device in place on the left arm of the user, according to a second embodiment,

FIG. 6 illustrates a buckle configured to be used in co-operation with the arm band, according to the second embodiment

FIG. 7 shows an exemplary block diagram of the device,

FIG. 8 shows a timing chart illustrating the method, at the heartbeat timescale,

FIG. 9 shows a timing chart illustrating the method at a larger timescale,

FIG. 10 illustrates the mechanical configuration of the device of the first embodiment in a stowed configuration,

FIG. 11 illustrates a general perspective view of the device of the first embodiment,

FIG. 12 illustrates a general overview of the disclosed method,

FIG. 13 shows steps of the method regarding pressure signals handling,

FIG. 14 shows step of the method regarding pulse transit time handling,

FIG. 15 shows a detailed sectional view of the armband.

### DETAILED DESCRIPTION OF THE DISCLOSURE

In the figures, the same references denote identical or similar elements. For clarity purposes, some parts are represented intentionally not at scale with regard to other parts. Also, some parts of timing charts can be represented intentionally not at scale.

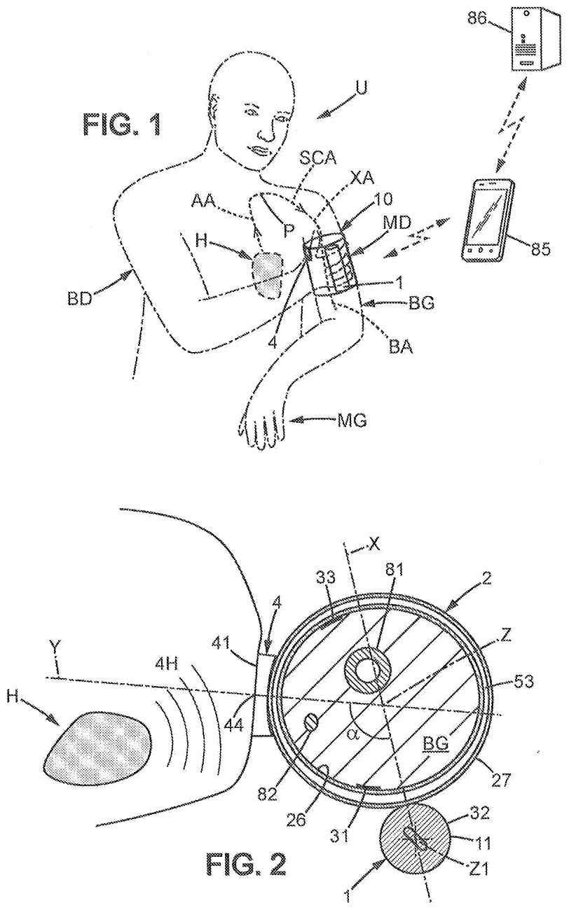

FIG. 1 shows an individual (also ‘user’) U in a configuration where he/she is using a device 10 according to the present disclosure. The device (otherwise called “apparatus”) 10 looks like a known brachial blood pressure sensing device (commonly named Blood Pressure Monitor i.e. in short “BP Monitor”), but the device exhibits extended functionalities as will be apparent below, so that the device can be called ‘upgraded BP Monitor.

The user U in question has, among other organs and limbs in his/her anatomy a left arm BG, a right arm BD, a heart H, a left hand MG a right hand MD.

Further the user in question has an aortic artery (‘aortic arch’) denoted AA, a subclavian artery SCA, an axillary artery XA, a brachial artery BA, belonging generally to the cardiovascular system of the user. Therefore a blood path of interest noted P is defined as the fluid conduit from the heart H to a reference point at the brachial artery BA.

Besides the left arm brachial artery as reference point, it should be understood that the proposed method would properly work with another predefined position where a hand can be secured around a limb, either a lower limb or an upper limb.

The device 10 has a wireless communication capability to exchange data with a mobile entity like a smartphone 85 (more generally a mobile device belonging to the users like a tablet, a laptop . . . ). Such smartphone 85 may in turn exchange data with a remote entity like an Internet server 86 (more generally any resource available somewhere in Internet, not excluding a so-called “cloud” resource).

The device 10 has either a small display or no display at all, since the user interface capability provided by the smartphone 85 is fully relevant to support displays relating to the use and extended functionalities of the device.

The device 10 is intended to be used at a home environment, for healthy people as well as people suffering from some disease, it may be used in a medical environment but is particularly suitable to be used by non-medical personnel, i.e. the user under measurement him/herself.

In the shown example, the device 10 comprises an armband 2 wrapped around the left arm BG, a control unit assembly 1, and an acoustic sensor denoted 4.

The rest of the time the device is stowed, notably in a folded configuration as will be seen later.

As illustrated on FIG. 1, the device comprises an armband wrapped around the arm i.e. the part of the upper limb comprised between the shoulder and the elbow. However it is not excluded to use the device elsewhere, at the forearm for example.

As illustrated on FIG. 1, the device is installed on the left arm of the user. However it is not excluded to use the device elsewhere, at the right arm for example.

As illustrated on FIG. 2, the left arm of the user includes a bone named humerus denoted 81, muscles (not specially shown), and the abovementioned brachial artery denoted 82. The humerus extends along an axis denoted Z. The armband band 2, when wrapped around the arm BG, has a general cylindrical shape with a reference axis substantially coinciding with arm axis Z.

The armband has an internal wall denoted 26 intended to contact the arm's skin and to press against the arm. The armband has an external wall denoted 27 on the opposite side of the band with regard to the internal wall 26.

In use configuration, the acoustic sensor 4 is located against the chest, i.e. against the left side of chest. Sound waves 4H emitted by the heart arc sensed by a sensitive portion 41 of the acoustic sensor 4, the sensitive portion 41 bearing on the left-side chest, i.e. adjacent to the chest. Handling of electrical signals transduced from acoustic waves 4H will be detailed later. It should be noted that acoustic waves 4H can be sensed by the sensitive portion 41 without trouble through a light clothing, an underwear or the like.

According to one particular option, the device is further equipped with an ECG function, i.e. ElectroCardioGraphic function.

For this purpose there are provided three contact electrodes 31, 32, 33, the three of them integrated in the device, without the need to have linking wires like in most prior art devices.

The first electrode 31 is arranged on the internal wall 26 of the band and has a sensitive face oriented toward the skin of the arm. The first electrode 33 is also arranged on the internal wall 26 and has also a sensitive face oriented toward the skin of the arm.

Each of the electrodes is formed as a thin pad of a surface comprised between 5 cm² and 10 cm²; for example, the shape of the thin pad is somewhat arched to follow the standard curvature of the skin of the arm.

In a particular option, first and third contact electrodes 31, 33 are disposed at distance from each other. Alternatively, first and third contact electrodes 31, 33 can be arranged differently, for example one above the other or one aside the other.

Whenever the armband is pressurized, first and third contact electrodes 31, 33 are firmly pressed against the skin of the arm, thereby ensuring a fairly good contact with a small electrical contact resistance. It should be noted that no gel is required at the contact electrode contrary to conventional habits. Contact electrodes are to be placed against the bare skin; however, thanks to the pressure, it is not excluded to have a light underwear cloth between the skin and the electrodes.

The contact electrodes can be made of stainless steel, silver, or other coated materials (coated with silver, chromium, gold, titanium or platinum), not excluding materials coated by physical vapor deposition technique (known as PVD techniques).

It is to be noted that two electrodes might be sufficient, therefore the third electrode 33 is considered optional.

Regarding the second electrode 32, it is arranged around the external surface of the control unit assembly as best seen at FIG. 11. A conductive material forms a coating of at least a part of the control unit housing. A metallic coating material (silver, titanium, chromium), are deposited by physical vapor deposition technique (known as PVD techniques).

The second electrode covers the lower third of the cylinder, for example all around the accessible circumference by the fingers of the user (see FIG. 1). Therefore, it is easy for the user to grab/seize the second electrode with a good electrical contact.

The above mentioned control unit assembly 1 has, in the shown example, an overall cylindrical shape with an axis denoted Z1 (cf FIGS. 2, 4, 11). The control unit assembly 1 is fixed to the arm band 2. For example, it is fixed to the first portion 21 as explained below.

As seen on Figures, the general arrangement is as follows: the control unit assembly 1 extends from the external wall 27 of the band with regard to the main axis Z along a direction denoted X. In use configuration, when the chest of the user is nearly vertical, X is substantially horizontal and in a front-rear direction.

In the illustrated case of a cylinder, the diameter of the control unit assembly 1, denoted D1, is less than 40 mm, for example about 35 mm or even about 30 mm.

The acoustic sensor 4 has a center 44, which is referred to to define the transversal axis Y, extends from the external wall 27 of the band with regard to the main axis Z and passing through the center 44 of the acoustic head. In use configuration, when the chest of the user is nearly vertical, Y is substantially horizontal and in a left-right direction.

Angular distance between axis X and Y is denoted by angle α. In use configuration, the angle α is comprised between 90° and 140°, for example between 110° and 130°. As shown at FIG. 1, in use configuration, the right hand can conveniently seized the control unit assembly 1 and the acoustic sensor is naturally placed against the chest of the user U.

Regarding the acoustic sensor 4, according to one preferred option, it is formed as a piezoelectric transducer, which can provide a very thin configuration; this piezoelectric transducer requires very little space projecting from the external wall 27 of the arm band; this piezoelectric transducer requires no electronic supply, no local electronic adaptation circuit.

Since the user naturally squeezes the acoustic sensor against the chest, the acoustic sensor can properly work through a thin cloth like a T-shirt, a shirt, even two layers of such cloth.

According to an alternative option, the acoustic sensor can be formed as a microphone.

In FIG. 2, a generic view of the armband is represented; this, type of band can be a ring adaptable in diameter/circumference. This kind of band can be inserted from the hand without opening the ring, and slid up to the shown position on the arm. There may be provided restriction means to decrease the play and lock the current position, before pneumatic inflation.

An inflatable bladder 53 is provided. Such a compliant inflatable bladder is known per se in blood pressure sensing apparatuses, and therefore not described in details here. At rest, the bladder is arranged within the thickness of the band, as an internal layer.

There may be provided an armature 25, otherwise called cuff holder, for structural support of at least a part of the band. The armature can be an arcuate plastic part, made from a plastic material with good or high mechanical properties (polypropylene, ABS, PVC, or the like), having a part-of-a-cylinder shape, or generally an arcuate shape.

According to one particular aspect, both the bladder 53 and the armature 25 extend circumferentially along the major part of the active portion of the armband 2; such that in use, the bladder is surrounding practically all the circumference of the aim of the user. Therefore, a homogeneous pressure is applied all around the arm which is beneficial for the accuracy of the measurement and the quality of the contact of the ECG electrodes 31, 33.

As visible on FIG. 7, the control unit assembly 1 comprises a battery 17, an electronic controller 6 and a pneumatic unit 5. We note that no external wired connection is needed.

The pneumatic unit 5 comprises at least a pump 7 driven by an electric motor 57, a release valve 56 also called “bleeder valve” 56, and a pressure sensor 61.

The pneumatic unit 5 may optionally comprise a check valve 58. The release valve 56 may be an On/off valve or a progressive valve.

The control unit assembly 1 comprises an On/Off switch 16; the user may start a measurement, after having installed the armband, by actuating the switch 16, pressing or touching according to various possible types of switches.

Further the control unit assembly 1 may further comprise a wireless interface 68 such as for example a wireless coupler (WiFi, Bluetooth™, BLE or the like), and a display 67 already mentioned. The display 67 can be a LED display and or a dot matrix display; on this display, blood pressure results can be displayed directly without use of the smartphone application.

There is provided a pneumatic hose 59 to fluidly connect the output of the pump to the bladder. It can be a one-way pneumatic connection or a two-ways pneumatic connection (59, 59′). According to one variant, there is provided a specific sense line 59′ decoupled from the pressurization line 59.

The pressure sensor 61 is in fluid connection with the inflatable bladder 53 through the specific line 59′, or through the common single 59 where applicable.

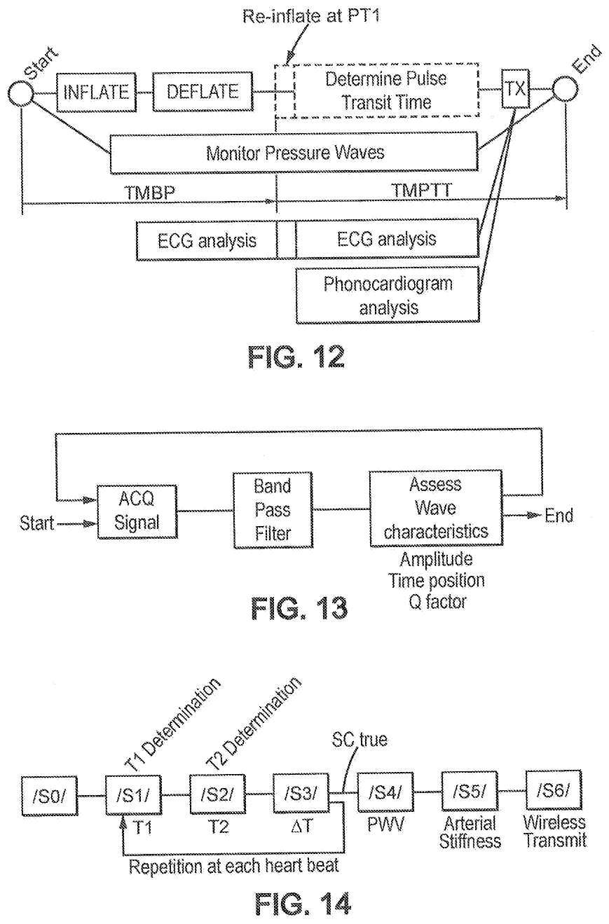

A first overview of the functionality of the device is given here, whereas it would be described later in more detail with the help of FIGS. 9 and 12.

A blood pressure measuring cycle is carried out first, and optionally, thereafter a pulse transit time PTT measuring cycle is carried out. In the same timeframe or separately, individual ECG signal analysis and/or phonocardiogram signal analysis can also be performed.

For the blood pressure measuring cycle, the electronic controller 6 is configured to first inflate the inflatable bladder 53 until the blood flow is greatly reduced by the pressure exerted on the arm. During inflation, the analysis of the evolution of pressure signals allows to infer the systolic pressure and the diastolic pressure. The controller is configured to then progressively deflate the bladder 53. The progressive reinstatement of the blood pressure waves is also analyzed by the electronic controller 6 to infer the systolic pressure and the diastolic pressure, in confirmation or replacement of values deduced during the inflation phase.

Regarding the pulse transit time PTT, the electronic controller 6 determines a first characteristic instant and a second characteristic instant, and the resulting time difference is used to calculate a pulse wave velocity to finally issue an index representative of the arterial stiffness to the user.

Now are described in detail embodiments and variants of the device structure.

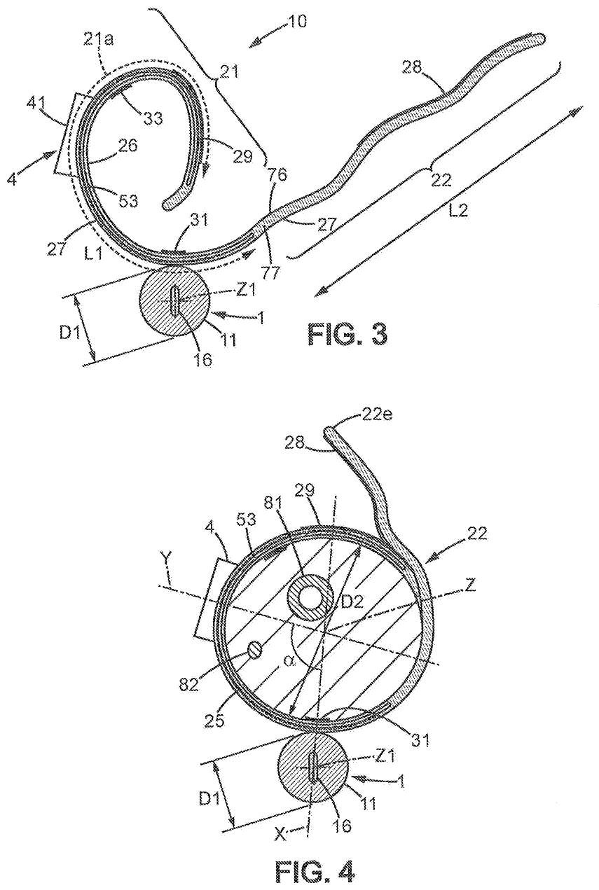

According to the first embodiment, illustrated at FIGS. 3, 4, 10 and 11, the band 2 comprises a first portion 21 and a second portion 22. The first portion 21 can be considered as the main portion since the control unit assembly 1 and the acoustic sensor 4 are affixed to this first portion 21, and furthermore this first portion houses the inflatable bladder 53, and optionally, the structural elastic armature 25 already mentioned.

The first portion 21 has a developed length denoted L1. The second portion 22 has a developed length denoted L2. The band has a height H. Likewise, the armband 2 is made from a generally rectangular shape with a width corresponding to dimension H and a length corresponding to added dimensions L1+L2.

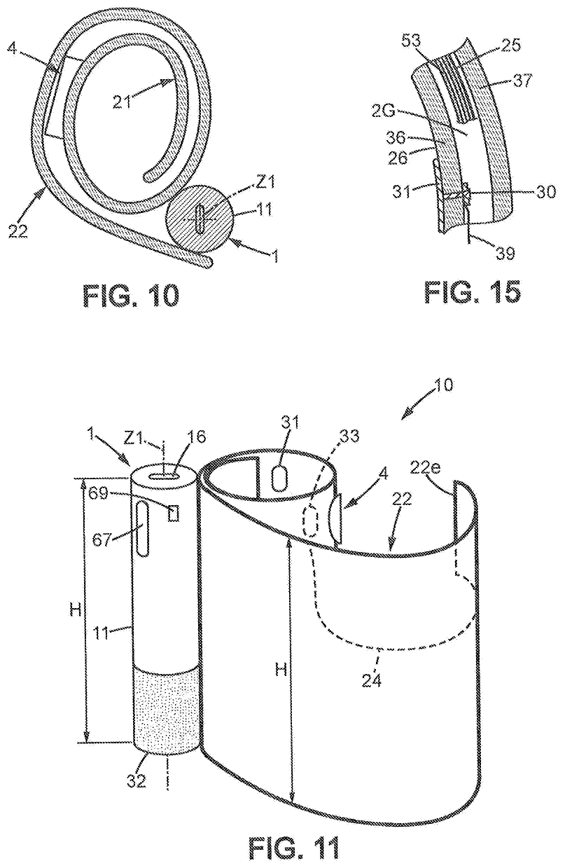

For installing the arm band 2 around the arm prior to inflating, the user starts for example from a stowed configuration depicted in FIG. 10, the user unrolls the second portion 22 (see FIG. 3) to make it possible to install the first portion around his/her left arm BG. Thanks to the elasticity of the first portion, the first portion 21 can be opened so as to facilitate the insertion of the arm into the internal space encompassed by the first portion. The user has to move away a little bit his/her arm from the chest to do that.

It should be noted that this configuration allows installing the band/cuff without inserting it along the forearm from the hand side.

Further operation involves the closing of the band 2 around the arm, and securing this configuration prior to inflation.

A hook pad 29 is arranged at the external wall of the first portion 21 and a corresponding loop pad 28 is arranged at the internal wall of the second portion 22. Of course the reverse configuration loop/hook is also possible.

Hook pad 29 and loop pad 28 may have a general rectangular shape. Regarding the dimensions, the hook pad 29 can extend along all the height H over a length of 5 cm to 10 cm; the loop pad 28 is longer, it also can extend along all the height 11 but over a length of 10 cm to 20 cm.

After the user has placed the first portion 21 around the arm on the side of the chest as described before, he/she pulls the second portion 22 toward the rear direction and then sticks the second portion 22 onto the first portion 21 by securing the loop pad 28 on the hook pad 29. The remaining unused end 22e of the second portion is left free.

Alternatively, according to a variant illustrated at FIG. 11, where a special cutout 24 is provided, the remaining unused end 22e of the second portion can be folded around back into the front direction.

FIG. 4 shows the device ready for inflation, the user presses the device onto his/her chest to guarantee acoustic adaptation, namely proper collection of acoustic waves.

It should be noted that the length of the second portion together with the length of the hook pad and the length of the loop pad allows the device to be adjusted to a large variety of arm circumference and diameter D2.

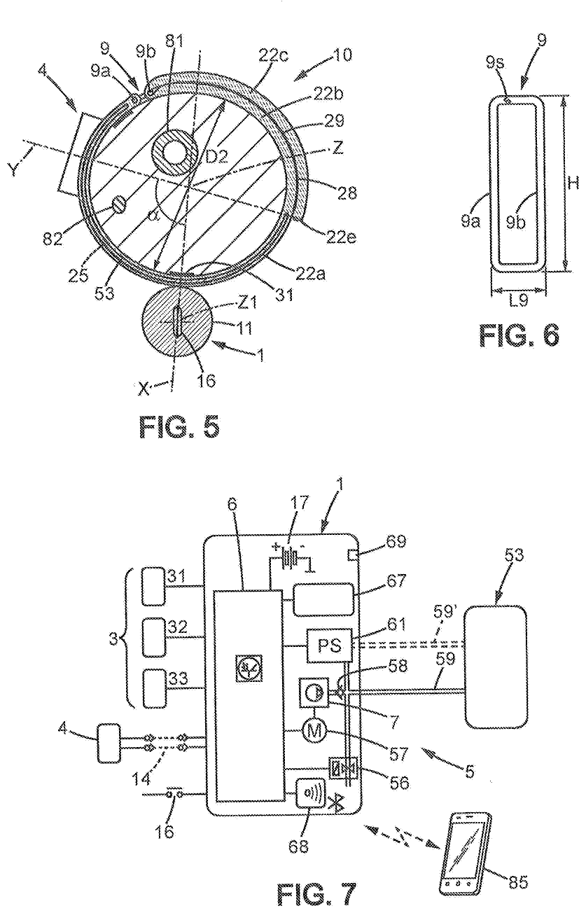

At FIGS. 5 and 6, a second embodiment of the armband is represented, which also allows to install the band/cuff without inserting it from the hand. Again, the band 2 comprises a first portion 21 and a second portion 22.

In this embodiment, the device also comprises a buckle 9, also named ‘return buckle’ as seen on FIG. 6. The buckle can be manufactured as a bent metallic wire, shaped as a rectangular loop. The buckle can also be made from plastic material with good or high mechanical properties. Two long sides 9a, 9b are connected at their respective ends by two small sides, thereby forming a rectangle with a length/height H and a width denoted L9.

The buckle is fastened to one circumferential end 21a of the first portion 21, at complete opposite from the other end 22e of the band 2.

For the assembly of the buckle on to the end 21a, it can be provide a seam in the band end, done after insertion of the loop buckle. Alternatively, the loop formed by the buckle may have an openable slit 9s, with self locking retaining means (hook and the like).

For installing the arm band 2 around the arm prior to inflating, the user places the first portion 21 around the arm on the side of the chest and passes the second portion 22 into the buckle 9 toward the rear and then pulls the end of the second portion 22 toward the front direction, until the arm wraps without substantial play the arm. Then the user sticks the returning portion 22c against the base 22b of the second portion to attach the attachment means, which results in the configuration shown at FIG. 5.

According to an aspect which is common to above embodiments, at least in part of the band, as shown at FIG. 15, the band 2 comprises an internal layer 36 and an external layer 37 with an interval 2G available between the two layers. In the gap 2G, there may be provided the bladder 53, the armature 25, rivets 30 for fastening the contact electrode 31 and a connection wire 39. Other wires may be present, for connecting the acoustic sensor to the electronic controller 6, and the third electrode.

There may be a single layer portion (i.e. without gap or interval), notably in the distal end of the second portion 22.

The band 2 is for example made of strong fabric, woven or on-woven or synthetic material.

Generally speaking, the band 2 comprises attachment means, for fixing the size of the armband prior to inflating. The attaching means may comprise one or more couple(s) of loop and hook pads.

Therefore, a ‘continuous’ unquantized adjustment of encompassed circumference is made available, for any size of arm.

It should be noted that in the present specification, arm circumference CIRC or diameter D2 are indifferently used, since we know that CIRC=π×D2.

According to other possible solutions, there may be provided attaching means including a releasable ratchet system, or a teeth system.

Regarding dimensions, the following preferences can be noted.

For a baseline device intended to encompass arms having a perimeter/circumference comprised between 20 cm and 42 cm, which represents most of the users

L1 is for example comprised between 20 cm and 32 cm.

L2 is for example comprised between 15 cm and 25 cm.

H is for example comprised between 12 cm and 16 cm.

For an XXL device (special large dimension variant) intended to encompass arms having a perimeter comprised between 40 cm and 62 cm

L1 is for example comprised between 40 cm and 45 cm.

L2 is for example comprised between 20 cm and 25 cm.

H is for example comprised between 14 cm and 18 cm.

This XXL variant can have a frusto-conical configuration, smaller diameter at the elbow oriented end.

When the device is unused, as illustrated at FIG. 10, it can be rolled up, whereby its size is less than 10 cm×10 cm×H.

In the variant illustrated at FIG. 11, the second portion 22 of the armband can exhibit a smaller height for its most distal area; this option is delineated by the dotted line 24; in this case, when the free end 22e of the second portion is wrapped around the first portion at the location of the acoustic sensor, the acoustic sensor 4 is still uncovered by the second portion. Therefore, even though the user installs the device by wrapping it ail around, this won't prevent the device from working regarding the acoustic acquisition against the user's chest.

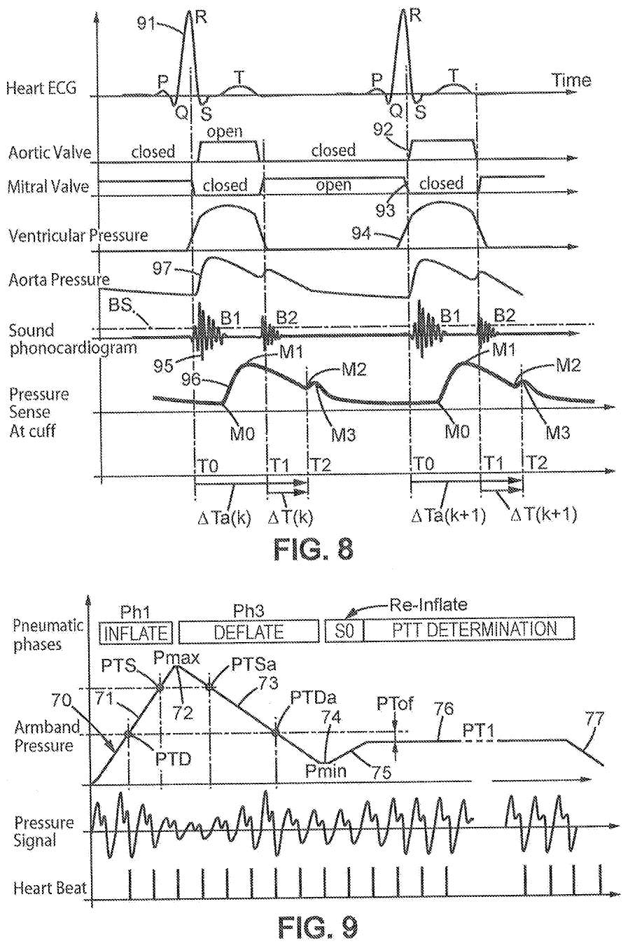

The device works as follows, as illustrated at FIGS. 8, 9, 12, 13 and 14.

During a measurement, the patient's heart generates electrical impulses that pass through the body at high speed. Also simultaneously the patient's heart generates acoustic waves that pass through the body with a certain sonic speed.

These impulses/waves accompany each heartbeat, and the heartbeat generates a pressure wave in artery network that propagates through the patient's vasculature at a significantly slower speed. The blood path P of interest has a certain length, let's say 30 cm to 40 cm according to the physical characteristic of the individual under measurement. As will be seen in more detail later, this length depends at first order on user's height denoted UH.

Immediately after the heartbeat ventricular contraction, the pressure wave leaves the heart and aorta, passes through the subclavian artery, to the brachial artery along the path P.

The ECG electrodes measure electrical signals which pass to an amplifier/filter circuit within the control unit assembly. For example, a filtering circuit is provided before the signal is digitized and entered into the microcontroller.

Within the controller, the signals are processed with an analog-to-digital converter to form the ECG digitized waveform and then recorded together with the time, of occurrence, namely instant T0. ECG waveform is named “QRS waveform” or “QRS complex” as sample shown at FIG. 8.

The acoustic waves are also band-pass filtered and amplified, for example after upfront digitalization. A bandpass filter with cutting frequencies [0.5 Hz-1 kHz] is applied, either in the analog font end before digitization or applied to the digitized acoustic signal.

“QRS waveform” 91 is the top curve shown on timechart at FIG. 8. This waveform is known per se thus not described in details here. Instant T0 corresponds in the illustrative embodiment to R apex, but another marker can be taken alternately.

Aortic valve open/close state is also shown just beneath, signal denoted 92.

Mitral valve open/dose state is also shown just beneath, signal denoted 93.

Just before aortic valve opening, the mitral valve closes; this produces a particular sound which is reflected in the first significant sound named B1 as shown on curve 95.

Further, after closing mitral valve and opening aortic valve, the ventricular volume decreases as blood is ejected to the aorta. At the same time ventricular pressure 94 exhibits a rounded apex. Aortic pressure curve is shown and denoted 97.

Sound phonocardiogram corresponds to curve denoted 95 electrically reflects waves received at the acoustic sensor 4.

This curve 95 exhibits two characteristics sounds; the first sound denoted B1 corresponds to the closing of the mitral valve, the second sound denoted B2 corresponds to the closing of the aortic valve.

A “significant sound”, in the sense of the present disclosure, is defined whenever a instantaneous power of the acoustic signals exceeds a predetermined threshold (BS), of FIG. 8.

It should be noted that sounds B1 and B2 exceeds BS threshold.

Pressure wave at pressure sensor 61 in fluid communication with bladder 53 is shown at curve 96.

This curve 96 exhibits three characteristics apexes. The first apex denoted M1 is a maximum apex; the second apex denoted M2 is a minimum local apex; the third apex denoted M3 is a maximum local apex.

Besides M0 is the minimum value, just before the rise which is a consequence/response of the arrival of the pressure pulse at the arm.

The second apex denoted M2 is a marker corresponding to arrival of the effect of the closure of aortic valve at the brachial artery within the arm band.

There may be defined a reference point in the arm, so that the ideal position for the device can be notified to the user, for example distance from the elbow internal fold, or another criterion.

Generally speaking, for the purpose of PTT, T1 is defined as the instant of the maximum instantaneous signal power of the second sound B2 reflecting when aortic valve doses.

Generally speaking, T2 is defined as the instant when second apex denoted M2 occurs.

Blood Pressure Procedure

This procedure is known in the art, and thus it is not described in details here. Basically, it comprises the following phases:
/Ph1/—start inflating the bladder, inflation phase is denoted 71 at FIG. 9, ‘INFLATE’ at FIG. 12,
/PhD/—determining Diastolic Blood pressure (PTD) during inflating phase
/PhS/—determining Systolic Blood pressure (PTS), during inflating phase
/Ph2/—stop inflating the bladder 72 (when hardly no more pressure wave is identified),
/Ph3/—start deflating the bladder, deflation phase is denoted 73 ‘DEFLATE ’ at FIG. 12,
/PhS/—determining Systolic Blood pressure (PTSa), during deflating phase
/PhD/—determining Diastolic Blood pressure (PTDa) during deflating phase

More precisely, the shape of the pressure waves are analyzed by the electronic controller. During inflation 71, as shown at FIG. 9, the shape of the pressure waves evolves, and a predefined criteria on the waveform makes the decision to record a first diastolic blood pressure PTD, and another set of predefined criteria on the waveform makes the decision to record a systolic blood pressure PTS.

In a similar manner, during deflation phase, the shape of the pressure waves are analyzed by the electronic controller. During deflation 73 the shape of the pressure waves evolves, and a predefined criteria on the waveform makes the decision to record another systolic blood pressure PTSa, and another set of predefined criteria on the waveform makes the decision to record another diastolic blood pressure PTDa.

It should be noted that pressure curved is shown after rectifying at FIG. 8, whereas it is shown before rectifying at FIG. 9.

The second systolic blood pressure noted PTSa can be regarded as a continuation of the first systolic blood pressure noted PTS. According to one example, an outputted systolic blood pressure can be an average of the value PTS and PTSa.

Similarly, the second diastolic blood pressure noted PTDa can be regarded as a confirmation of the first diastolic blood pressure noted PTD. According to one example, an outputted diastolic blood pressure can be an average of the value PTD and PTDa.

PTT Procedure

This procedure is adapted to determine as accurately as possible the pulse transit time PTT.

It comprises the following phases:
/S0/—inflate 75 the bladder at a predetermined pressure denoted PT1, the inflation bringing the pressure from the lowest value 74 to this predefined level PT1, detailed below
/S1/—determining the above-mentioned aortic valve closing instant T1(k) from acoustic signals,
/S2/—determining subsequently, from pressure signals, a characteristic point (M2) of the pressure signal curve occurring at instant T2(k),
/S3/—calculate a time difference, defined as ΔT(k)=T2(k)−T1(k)

The predetermined pressure PT1 can be defined as a function of PTD, for example with, a value below the diastolic pressure PTD; this value may be defined by a predefined offset PTof; in other words, PT1 can be such PT1=PTD−PTof, with PTof for example equal to 10 mmHg (10 Torr).

According to one option, said characteristic point (M2) is the above-mentioned local minimum apex, after first apex M1.

Further, steps S1 to S3 are repeated until a stop criterion SC is met. This stop criterion SC can be defined according to different possibilities. One consists in predefined duration. Another one consists in counting the number N of heartbeat cycle; for example N is chosen between 4 and 20, for example between 6 and 12.

Overall duration for BP procedure is denoted TMBP and duration for pulse transit time procedure is denoted TMPTT. For example TMBP is less than 10 seconds. For example TMPTT is less than 12 seconds. According to a user configuration, selectively chosen by user through the smartphone or locally by a double press on the switch 16, the user can choose to carry out only the blood pressure or BP procedure plus PTT procedure.

During PTT procedure, pump 7 is not energized and a bleeder valve comprised in the pneumatic unit is not energized. Therefore, no intrinsic parasitic noise disturbs the phonocardiogram analysis and therefore the PTT procedure and related calculation is not disturbed by endogenous noise.

The electronic controller 6 computes therefrom a time difference, defined as

ΔT(k)=T2(k)−T1(k) for the heartbeat arbitrarily numbered k.

PWV is the wave velocity along path P.

PWV is defined as PWV=length(P)/ΔT(k)

In practice, we prefer to rely on a successive series of N measurements; in this condition, the method may comprise the following:
/S41/—calculate an average value ΔTav of ΔT(k), for k=j to j+N
/S42/—calculate a Pulse Wave Velocity (PWV) defined as PWV(k)=length(P)/ΔTav,

The number N of effective PTT measurement can be chosen between 4 and 20.

The average value can be computed as follows

$$Δ Tav = \frac{1}{N} {\sum }_{k = j + 1}^{k = j + N} Δ T ( k ) .$$

The height (UH) of the user is taken into account at step S42, namely length(P)=F1 (UH)

Also any combination of age (UA), gender (UG), and weight (UW), of the user may additionally be taken into account at step /S42/, namely length(P)=F2 (UH,UA,UG,UW).

Arterial stiffness AS can be defined as a function of PWV. AS can be expressed as a rating between 1 and 10; it can also be expressed as an equivalent age of the person. Arterial stiffness AS is referred to as step /S5/.

FIG. 14 shows a summary of the above mentioned steps /S0/ to /S5/, with a repetition until stop criterion (SC) becomes true (in other words criterion is met).

An optional step referred to as step /S6/ consists in transmitting the resulting data and parameters to the smartphone 85 over the wireless link. Please note that alternatively this transmission can be continuous all along the process, ECG signal and/or phonocardiogram can be displayed in real time on the smartphone application. Transmission of resulting data and parameters can also be done by packets, for example one packet after blood pressure values are obtained (BP procedure) and another packet after PTT Procedure.

A plurality of subsequent evaluations of Pulse Wave Velocity are recorded for a particular user to form a history curve, and a deviation in said curve is notified to said particular user.

It should be noted that the disclosed device has complete power autonomy; According to a particular option, the device is formed as an integral unit, there is no external wire, not external hose, therefore style and practicality are enhanced.

Summary of Cardiovascular Parameters/Functions that can be Monitored by the Device:

As already mentioned, Diastolic blood pressure PTD, and systolic blood pressure PTS can be obtained from the blood pressure procedure, as a motion of blood pressure cuff.

Also, as described before, PTT procedure allows to determine the arterial stiffness.

Also, heart rate HR can be inferred from ECG signals 91, front phonocardiogram signals 95, or from oscillometric signals 96, by measuring the average time separation between two heartbeats and deducing therefrom the number of heartbeat and minute.

HR variability is also calculated, over at least three subsequent heartbeats.

Also, the device can perform ECG signal analysis, for detection of arrhythmia, as known per se. Some anomalies in ECG signal 91 can be identified and associated to a type of arrhythmia, such as arterial fibrillation.

Also, the device can perform a phonocardiogram analysis for detection of heart anomalies responsible for so-called heart murmurs. In particular, some anomalies in phonocardiogram signal 95 can be identified and associated to a valvulopathy, concerning any of the mitral, tricupside, aortic, or pulmonary valves.

ECG analysis and phonocardiogram analysis can be carried out simultaneously with the PTT procedure, or separately, namely before or after PTT procedure.

As illustrated at FIG. 12, ECU analysis can be performed all along and at any time. According to one option, though ECG analysis is suspended whenever the electric motor is energized to run the pump (this produce electromagnetic interference detriment as to ECU signals).

Regarding the phonocardiogram analysis, it is for example carried out when no intrinsic source of noise is present, for example during the constant pressure sequence for PTT determination.

As shown at FIG. 13, pressure signals are sensed by pressure sensor 61 and made available at the microcontroller 6. According to one option the microcontroller 6 digitizes the signal and then applies a digital bandpass filter. For example a band range of [0.3 Hz-3 kHz] or [0.5 Hz-1 kHz] can be used.

The microcontroller 6 identifies wave pulses and determines values characteristics of said pulses, i.e. amplitude, time position, apexes, derivatives, Q-factor, . . . .

System, Application and Remote Patient Monitoring

The user can follow his/her own metrics on a smartphone application.

The device can be used by more than one user, selection of relevant user can be done through the local display 67 or through the smartphone application. When actuated, directly on the device, the activation switch 16 can be used to scroll across several users names.

There is provided a Micro USB connection 69 for battery recharge and for up/downloading of data.

The smartphone application can display a history of measurement reports, which can include BP, PTT, ECG signals, phonocardiograms.

It should be noted that phonocardiogram can be replayed via the smartphone application and the loudspeaker/headphones coupled to the smartphone.

Smartphone application can issue time reminders for the user, so the user can measure cardiovascular parameters regularly with the integrated device 10.

The integrated device 10 is configured to send an alert to the physician whenever some particular thresholds are exceeded or when particular events are detected, such as an episode of arrhythmia. Such thresholds regarding blood pressure can be defined either by the user him/herself or by the physician/doctor.

The user can add the contextual note(s), such as medicine intake or a life circumstance (just after wakeup, just before going to bed), to one or more measurement reports.

Measurement reports can be sent remotely to a server that can be accessed the physician/doctor, so that the physician can analyze the patient's data from a distant location.

The system allows remote analysis of ECG signals; the user can receive in return the diagnostic from the physician.

Although the drawings and the text above mainly focus on an example with an armband placed around the left arm, it should be understood that the predefined position where pressure signals are captured could also be somewhere else on one limb of the user, for example at the forearm, at the wrist, somewhere else on the right arm of the user, including the wrist; it is not excluded to implement the proposed method on a lower limb of the user. Of course, parameters to be used are to be adapted in particular to the length of the arterial blood path P.

Also, it is important to note that the acoustic sensor can be placed somewhere else other than the left side chest of the user, the acoustic sensor may be provided with an extension cord to electrically couple the acoustic sensor to the control unit.

Additionally, there may be provided a control unit implemented differently, not necessarily adjacent to the armband.

According to another embodiment, instead of an inflatable bladder, the system may include pressure exerting means that are different from compressed air. For example, it can be two bladders filled with water with a ballasting system. As per another example, it can be a mechanically collar with controllable restraint. Although, solutions like a set of piezoelectric actuators, or a collar with temperature dependent memory alloys are not excluded.

According to still another embodiment, instead of a pressure sensor, at least one blood circulation parameter like the instantaneous local speed can be sensed by a Doppler effect sensor placed adjacent to a surface blood vessel.

Therefore, there is defined under the generic term “blood circulation sensor” either a pressure sensor as mentioned in the text and drawings, or a Doppler effect sensor as mentioned just above, without excluding the case of other types of sensors like a photoplethysmography sensor placed next to a skin portion with a blood vessel.

The blood circulation sensor delivers signals that are representative of instantaneous blood circulation parameters prevailing at the sensed position, i.e. the blood circulation sensor delivers signals that are an image of instantaneous blood circulation parameters prevailing at the sensed position.

1. A method to collect cardiovascular data relating to an individual user (U), in a system comprising an acoustic sensor configured to be coupled to the chest of the user, a blood circulation sensor configured to be placed at a predefined position at the user's body, an arterial blood path (P) being defined from the heart to the predefined position, the method comprising a set of steps named PTT procedure:
/A1/—acquiring acoustic signals at the acoustic sensor,
/A2/—acquiring of blood circulation signals at the blood circulation sensor, said blood circulation signals being representative of instantaneous blood circulation parameters prevailing at the predefined position,
/S1/—determining an aortic valve closing instant T1(k) from the acoustic signals,
/S2/—determining subsequently, from the blood circulation signals, a characteristic point (M2) occurring at an instant T2(k),
/S3/—calculate a time difference, defined as ΔT(k)=T2(k)−T1(k)
/Sloop/—repeating, for each heartbeat, steps /S1/ to /S3/ until a stop criterion (SC) is met.

2. The method according to claim 1, wherein the system comprises means for exerting pressure around a limb of the user, at the predefined position, and the blood sensor may be a pressure sensor, wherein blood fluid circulation parameters signals are pressure signals, and wherein the method comprises, prior to step /S1/:
/S0/—exert a predetermined pressure (PT1)
And at step /S2/ the characteristic point (M2) is determined from a pressure signal curve.

3. The method according to claim 2, wherein the means for exerting pressure is a band having an inflatable bladder configured to be placed at the predefined position, a pneumatic unit with at least a pump, the pressure sensor being configured to be fluidly connected to the inflatable bladder.

4. The method according to claim 3, wherein, the predefined position is at the left arm (BG) of the user, the band is an arm band and is configured to be placed around the left arm of the user.

5. The method according to claim 2, wherein the characteristic point (M2) of the pressure signal curve is defined as a succession of a maximum apex (M1) and a minimum apex (M2), said instant T2(k) being defined as the instant when said minimum apex occurs.

6. The method according to claim 1, wherein the system comprises a set of contact electrodes for electrocardiographic sensing, configured to be brought in contact with the skin of the user U, the method comprising the steps:
/A3/—acquiring ECG signals at the contact electrodes,
/S10/—determining a characteristic QRS signal from ECG signals as a synchronization signal (T0) reflecting heartbeat.

7. The method according to claim 1, wherein the aortic valve closing instant T1(k) is defined as a second significant sound (B2) of the heartbeat.

8. The method according to claim 7, wherein at step /S1/, the aortic valve closing instant T1(k) is determined as follows:
/S11/—identifying a first significant, sound (B1) of the heartbeat, reflecting mitral valve closing, just following QRS signal at T10,
/S12/—identifying a second significant sound (B2) of the heartbeat, reflecting aortic valve closing, and record said second significant sound (B2) as instant T1(k).

9. The method according to claim 1, wherein the method comprises, before the PTT procedure, a preliminary set of steps named Blood Pressure procedure comprising the steps:
/Ph1/—start inflating the bladder,
/Ph2/—stop inflating the bladder (when no more pressure wave is identified),
/Ph3/—start deflating the bladder,
meanwhile are performed the following steps:
/PhS/—determining Systolic Blood pressure (PTS), during inflating phase and/or deflating phase
/PhD/—determining Diastolic Blood pressure (PTD), during inflating phase and/or deflating phase.

10. The method according to claim 9, wherein prior to steps /S1/ to /S3/, the predetermined pressure PT1, providing pressurization for PTT procedure, is calculated with reference to the diastolic pressure (PTD) determined at step /PhD/.

11. The method according to claim 1, wherein during PTT procedure, pump is not energized and a bleeder valve comprised in the pneumatic unit is not energized.

12. The method according to claim 1, further comprising:
/S41/—calculate an average value ΔTav of ΔT(k), for k=j to j+N
/S42/—calculate a Pulse Wave Velocity (PWV) defined as PWV(k)=length (P)/ΔTav,
/S5/—asses therefrom an arterial stiffness (AS) of the user.

13. The method according to claim 12, wherein the height (UH) of the user is taken into account at step /S42/, namely length(P)=F1 (UH).

14. The method according to claim 12, wherein subsequent evaluations of Pulse Wave Velocity are recorded for a particular user (U) to form a history curve, and a deviation in said curve is notified to said particular user.

15. A system comprising an acoustic sensor configured to be coupled to the chest of the user, a blood circulation sensor configured to be placed at a predefined position at the user's body, an arterial blood path (P) being defined from the heart to the predefined position, characterized in that the system is configured to carry out the method comprising a set of steps named PTT procedure:
/A1/—acquiring acoustic signals at the acoustic sensor,
/A2/—acquiring of blood circulation signals at the blood circulation sensor, said blood circulation signals being representative of instantaneous blood circulation parameters prevailing at the predefined position,
/S1/—determining aortic valve closing instant T1(k) from acoustic signals,
/S2/—determining subsequently, from blood circulation signals, a characteristic point (M2) occurring at instant T2(k),
/S3/—calculate a time difference, defined as ΔT(k)=T2(k)−T1(k)
/Sloop/—repeating, for each heartbeat, steps /S1/ to /S3/ until a stop criterion (SC) is met.

16. A computer readable medium encoded with instructions that, when executed by a computer, cause performance of a method in a system comprising an acoustic sensor (4) configured to be coupled to the chest of the user, a blood circulation sensor configured to be placed at a predefined position at the user s body, an arterial blood path (P) being defined from the heart to the predefined position, the method comprising a set of steps named PTT procedure:
/A1/—acquiring acoustic signals at the acoustic sensor,
/A2/—acquiring of blood circulation signals at the blood circulation sensor, said blood circulation signals being representative of instantaneous blood circulation parameters prevailing at the predefined position,
/S1/—determining aortic valve closing instant T1(k) from acoustic signals,
/S2/—determining subsequently, from blood circulation signals, a characteristic point (M2) occurring at instant T2(k),
/S3/—calculate a time difference, defined as ΔT(k)=T2(k)−T1(k)
/Sloop/—repeating, for each heartbeat, steps /S1/ to /S3/ until a stop criterion (SC) is met.
